SEQUENCE LISTING (81 sequences)

SEQ ID NO: 1 - DNA, 30 bp, Artificial sequence
  rs3110496 forward primer
acgttggatgcacaaatcactgccgctctg

SEQ ID NO: 2 - DNA, 30 bp, Artificial sequence
  rs1017529 forward primer
acgttggatgccagggcaggatcataaatg

SEQ ID NO: 3 - DNA, 30 bp, Artificial sequence
  rs3744626 forward primer
acgttggatgtttctcccccgtgacctttc

SEQ ID NO: 4 - DNA, 30 bp, Artificial sequence
  rs3115095 forward primer
acgttggatgttaacctctctgagccactg

SEQ ID NO: 5 - DNA, 30 bp, Artificial sequence
  rs1128913 forward primer
acgttggatgaagcaacccccaagacaaag

SEQ ID NO: 6 - DNA, 30 bp, Artificial sequence
  rs894606 forward primer
acgttggatggccaattaataaacgctgcc

SEQ ID NO: 7 - DNA, 30 bp, Artificial sequence
  rs36053049 forward primer
acgttggatgaacacctcattcaaccagcc

SEQ ID NO: 8 - DNA, 30 bp, Artificial sequence
  rs3785960 forward primer
acgttggatgatacacatgcacgcatgcac

SEQ ID NO: 9 - DNA, 30 bp, Artificial sequence
  rs4795515 forward primer
acgttggatgagcgagaccccaactcttaa

SEQ ID NO: 10 - DNA, 30 bp, Artificial sequence
  rs35481649 forward primer
acgttggatgactctcgggcattaaagcgg

SEQ ID NO: 11 - DNA, 30 bp, Artificial sequence
  rs41274278 forward primer
acgttggatgctcctagcctcaataactgc

SEQ ID NO: 12 - DNA, 30 bp, Artificial sequence; identical to SEQ ID NO: 11
  rs55956743 forward primer

SEQ ID NO: 13 - DNA, 29 bp, Artificial sequence
  rs34131428 forward primer
acgttggatgtgcagcgagaggtgagggt

SEQ ID NO: 14 - DNA, 29 bp, Artificial sequence
  rs11548561 forward primer
acgttggatgaaagggcttcagggcagag

SEQ ID NO: 15 - DNA, 29 bp, Artificial sequence
  ENSSNP11225832 forward primer
acgttggatgaaagggcttcagggcagag

SEQ ID NO: 16 - DNA, 29 bp, Artificial sequence
  rs34015437 forward primer
acgttggatgagtgaagggcacagctgag

SEQ ID NO: 17 - DNA, 30 bp, Artificial sequence
  rs36109144 forward primer
acgttggatgggtggaagtcttcctctttg

SEQ ID NO: 18 - DNA, 30 bp, Artificial sequence
  rs550818 forward primer
acgttggatgtgaggctggaccttggactt

SEQ ID NO: 19 - DNA, 29 bp, Artificial sequence
  rs55778351 forward primer
acgttggatggaggtgcatggaatgtggg

SEQ ID NO: 20 - DNA, 30 bp, Artificial sequence
  rs41274276 forward primer
acgttggatgacccccatgagccagttcag

SEQ ID NO: 21 - DNA, 29 bp, Artificial sequence
  rs3211044 forward primer
acgttggatgtgcgtgttgagtgtgtggg

SEQ ID NO: 22 - DNA, 29 bp, Artificial sequence
  rs57467547 forward primer
acgttggatgtgcgtgttgagtgtgtggg

SEQ ID NO: 23 - DNA, 29 bp, Artificial sequence
  rs11548559 forward primer
acgttggatgtgcgtgttgagtgtgtggg

SEQ ID NO: 24 - DNA, 30 bp, Artificial sequence
  rs60006555 forward primer
acgttggatgctcgccccttctctatgaac

SEQ ID NO: 25 - DNA, 30 bp, Artificial sequence
  rs55677368 forward primer
acgttggatgacccaccaccacacaggtag

SEQ ID NO: 26 - DNA, 30 bp, Artificial sequence; identical to SEQ ID NO: 25
  rs11548562 forward primer

SEQ ID NO: 27 - DNA, 30 bp, Artificial sequence
  rs565977 forward primer
acgttggatgcgagaggaggtaactgttag

SEQ ID NO: 28 - DNA, 30 bp, Artificial sequence
  rs3110496 reverse primer
acgttggatgctcctgcttgttaagatggg

SEQ ID NO: 29 - DNA, 30 bp, Artificial sequence
  rs1017529 reverse primer
acgttggatgtatagtcagccttccatgcc

SEQ ID NO: 30 - DNA, 30 bp, Artificial sequence
  rs3744626 reverse primer
acgttggatgtctcaagggtggaggagatg

SEQ ID NO: 31 - DNA, 30 bp, Artificial sequence
  rs3115095 reverse primer
acgttggatgtgtgatgctgcttgcaactg

SEQ ID NO: 32 - DNA, 30 bp, Artificial sequence
  rs1128913 reverse primer
acgttggatgtgtgcacaaatgccagcatc

SEQ ID NO: 33 - DNA, 30 bp, Artificial sequence
  rs894606 reverse primer
acgttggatgagtatctaagcagggcacac

SEQ ID NO: 34 - DNA, 30 bp, Artificial sequence
  rs36053049 reverse primer
acgttggatggcttcagggatgtgactaac

SEQ ID NO: 35 - DNA, 30 bp, Artificial sequence
  rs3785960 reverse primer
acgttggatgtaaacccagacctcttccag

SEQ ID NO: 36 - DNA, 30 bp, Artificial sequence
  rs4795515 reverse primer
acgttggatgctggaaatggagatgacagc

SEQ ID NO: 37 - DNA, 29 bp, Artificial sequence
  rs35481649 reverse primer
acgttggatgcctgcgtcagtcataaggg

SEQ ID NO: 38 - DNA, 30 bp, Artificial sequence
  rs41274278 reverse primer
acgttggatgtctgcagagagagacctaag

SEQ ID NO: 39 - DNA, 30 bp, Artificial sequence; identical to SEQ ID NO: 38
  rs55956743 reverse primer

SEQ ID NO: 40 - DNA, 30 bp, Artificial sequence
  rs34131428 reverse primer
acgttggatgttctccgcctgcagcttgtg

SEQ ID NO: 41 - DNA, 29 bp, Artificial sequence
  rs11548561 reverse primer
acgttggatgagtgaacgggcggaacaca

SEQ ID NO: 42 - DNA, 29 bp, Artificial sequence
  ENSSNP11225832 reverse primer
acgttggatgagtgaacgggcggaacaca

SEQ ID NO: 43 - DNA, 29 bp, Artificial sequence
  rs34015437 reverse primer
acgttggatgcacactgactccatccagc

SEQ ID NO: 44 - DNA, 30 bp, Artificial sequence
  rs36109144 reverse primer
acgttggatggtctctgttttctggacctg

SEQ ID NO: 45 - DNA, 30 bp, Artificial sequence
  rs550818 reverse primer
acgttggatgaatcccatctgatcgctacc

SEQ ID NO: 46 - DNA, 30 bp, Artificial sequence
  rs55778351 reverse primer
acgttggatgttagtgctgccacactccct

SEQ ID NO: 47 - DNA, 30 bp, Artificial sequence
  rs41274276 reverse primer
acgttggatgggagactatgtacaaaggag

SEQ ID NO: 48 - DNA, 28 bp, Artificial sequence
  rs3211044 reverse primer
acgttggatgatcagggaggcagctggc

SEQ ID NO: 49 - DNA, 28 bp, Artificial sequence
  rs57467547 reverse primer
acgttggatgatcagggaggcagctggc

SEQ ID NO: 50 - DNA, 28 bp, Artificial sequence
  rs11548559 reverse primer
acgttggatgatcagggaggcagctggc

SEQ ID NO: 51 - DNA, 30 bp, Artificial sequence
  rs60006555 reverse primer
acgttggatgaccctagccagcacacattc

SEQ ID NO: 52 - DNA, 30 bp, Artificial sequence
  rs55677368 reverse primer
acgttggatgatcactacctgcacctgctg

SEQ ID NO: 53 - DNA, 30 bp, Artificial sequence; identical to SEQ ID NO: 52
  rs11548562 reverse primer

SEQ ID NO: 54 - DNA, 30 bp, Artificial sequence
  rs565977 reverse primer
acgttggatggggaatggaaaattcaaggg

SEQ ID NO: 55 - DNA, 20 bp, Artificial sequence
  rs3110496 extension reaction primer
tcactgccgctctggcaccg

SEQ ID NO: 56 - DNA, 22 bp, Artificial sequence
  rs1017529 extension reaction primer
gatctgaggtctgtggtcctac

SEQ ID NO: 57 - DNA, 25 bp, Artificial sequence
  rs3744626 extension reaction primer
ggacctttccgcagcctgagtcgta

SEQ ID NO: 58 - DNA, 22 bp, Artificial sequence
  rs3115095 extension reaction primer
cactgtttcttcatcacagagg

SEQ ID NO: 59 - DNA, 23 bp, Artificial sequence
  rs1128913 extension reaction primer
agacaaagtccaccccatcaagt

SEQ ID NO: 60 - DNA, 26 bp, Artificial sequence
  rs894606 extension reaction primer
caattaataaacgctgccgtcagtca

SEQ ID NO: 61 - DNA, 17 bp, Artificial sequence
  rs36053049 extension reaction primer
agcgcacgtgcctaatt

SEQ ID NO: 62 - DNA, 20 bp, Artificial sequence
  rs3785960 extension reaction primer
tagaatcagaccatagaagt

SEQ ID NO: 63 - DNA, 20 bp, Artificial sequence
  rs4795515 extension reaction primer
attatctaatcctccctccc

SEQ ID NO: 64 - DNA, 19 bp, Artificial sequence
  rs35481649 extension reaction primer
gggccagcttttgtcgccc

SEQ ID NO: 65 - DNA, 17 bp, Artificial sequence
  rs41274278 extension reaction primer
gcagggaggccaacacc

SEQ ID NO: 66 - DNA, 25 bp, Artificial sequence
  rs55956743 extension reaction primer
tcctagcctcaataactgccccctg

SEQ ID NO: 67 - DNA, 17 bp, Artificial sequence
  rs34131428 extension reaction primer
gacagcactttgcaccc

SEQ ID NO: 68 - DNA, 18 bp, Artificial sequence
  rs11548561 extension reaction primer
agggcagagccaggttca

SEQ ID NO: 69 - DNA, 18 bp, Artificial sequence
  ENSSNP11225832 extension reaction primer
ggcggaacacacacccat

SEQ ID NO: 70 - DNA, 21 bp, Artificial sequence
  rs34015437 extension reaction primer
agctgaggcagacaccccttt

SEQ ID NO: 71 - DNA, 17 bp, Artificial sequence
  rs36109144 extension reaction primer
aacctcttcccttccag

SEQ ID NO: 72 - DNA, 20 bp, Artificial sequence
  rs550818 extension reaction primer
ccaaccttggactttaagcc

SEQ ID NO: 73 - DNA, 30 bp, Artificial sequence; identical to SEQ ID NO: 46
  rs55778351 extension reaction primer

SEQ ID NO: 74 - DNA, 23 bp, Artificial sequence
  rs41274276 extension reaction primer
cccatgagccagttcagccctac

SEQ ID NO: 75 - DNA, 22 bp, Artificial sequence
  rs3211044 extension reaction primer
ggactgtccgggtagccagttt

SEQ ID NO: 76 - DNA, 17 bp, Artificial sequence
  rs57467547 extension reaction primer
agcttcccttccccacc

SEQ ID NO: 77 - DNA, 20 bp, Artificial sequence
  rs11548559 extension reaction primer
gatggaccaggcctccggaa

SEQ ID NO: 78 - DNA, 22 bp, Artificial sequence
  rs60006555 extension reaction primer
gaatcaaaaaccacttccctcc

SEQ ID NO: 79 - DNA, 19 bp, Artificial sequence
  rs55677368 extension reaction primer
cccagggaagaagcgggat

SEQ ID NO: 80 - DNA, 18 bp, Artificial sequence
  rs11548562 extension reaction primer
acagccttcccaggctgc

SEQ ID NO: 81 - DNA, 18 bp, Artificial sequence
  rs565977 extension reaction primer
cgaggaaggtggggacaa

(19) United States
(12) Patent Application Publication
(10) Pub. No.: US 2015/0322526 A1
(43) Pub. Date: Nov. 12, 2015

(54) COMPOSITION, KIT, AND METHOD FOR DIAGNOSING ADHD RISK

(71) Applicants: KOREA ADVANCED INSTITUTE OF SCIENCE AND TECHNOLOGY, Daejeon (KR); SNU R&DB FOUNDATION, Seoul (KR)

(72) Inventors: Changwon KANG, Daejeon (KR); Eunjoon KIM, Daejeon (KR); Jae-Won KIM, Seoul (KR); Eunjin KIM, Daejeon (KR); Hyejung WON, Daejeon (KR); Won MAH, Daejeon (KR); Soo-Churl CHO, Seoul (KR)

(21) Appl. No.: 14/795,188

(22) Filed: Jul. 9, 2015

Related U.S. Application Data
(62) Division of application No. 13/446,711, filed on Apr. 13, 2012.

(30) Foreign Application Priority Data
Sep. 20, 2011 (KR) 10-2011-0094918

(51) Int. Cl. C12Q 1/68

(52) U.S. Cl. CPC C12Q 1/6883; C12Q 2600/156

(57) ABSTRACT

Provided is a method for predicting the risk of attention deficit hyperactivity disorder (ADHD). The method comprises: 1) isolating a nucleic acid species from a biological species derived from a subject; 2) identifying the nucleotide of rs550818 single nucleotide polymorphism (SNP) in the GIT1 gene, which is the 24926101st nucleotide residue on human chromosome 17, from the nucleic acid isolated from stage 1); and 3) determining the risk of ADHD to be high when the genotype of rs550818 SNP that is identified in stage 2) carries T nucleotide; wherein the composition comprising a primer pair for amplifying a rs550818 single nucleotide polymorphism (SNP), wherein the primer pair comprises a primer having a sequence of SEQ ID NO:18 and a primer having a sequence of SEQ ID NO: 45.

### CROSS-REFERENCE TO RELATED APPLICATIONS

This application is a divisional application of U.S. Ser. No. 13/446,711, filed on Apr. 13, 2012, which claims priority under 35 U.S.C. §119 to Korean Patent Application No. 10-2011-0094918, filed on Sep. 20, 2011 in the Korean Intellectual Property Office, the disclosure of which is incorporated herein by reference in its entirety.

### TECHNICAL FIELD

The following disclosure relates to a technology of typing a particular single nucleotide polymorphism (SNP) having significant association with attention deficit hyperactivity disorder (ADHD) and using the SNP genotypes as a marker to predict the risk of ADHD, and more particularly to a composition for diagnosing ADHD risk, including a polynucleotide having C or T nucleotide at the 24926101st residue on human chromosome 17 and a method of predicting the risk of ADHD by identifying the nucleotide of rs5508181 SNP in the GIT1 gene. Further, the present invention relates to a composition for diagnosing the risk of ADHD, including a probe for typing the SNP or a primer for amplifying the chromosomal region, and a diagnosing kit having the probe immobilized on a surface thereof.

Therefore, the method of predicting, and the composition and the kit for diagnosing according to the following disclosure are useful technologies that can conveniently classify risk groups for ADHD at high sensitivity, so as to prevent in advance or early detect the risk groups.

### BACKGROUND

Attention deficit hyperactivity disorder (ADHD) is more common in childhood than adulthood, and is referred to as a state characterized by persistent inattention, hyperactivity, and impulsivity. When these symptoms are ignored without being treated, they lead to continuous difficulties in lives throughout childhood, and in many cases, these symptoms may linger even during the adolescence and adulthood (Barkey R. A., Attention deficit hyperactivity disorder. A Handbook for Diagnosis and Treatment, Guilford, N.Y., 2006).

It has been reported that neurochemical factors, genetic factors, and environmental factors are the causes of ADHD. Particularly, an association between dopamine, which is a neurotransmitter, and ADHD has been known (Swanson J M et al., Neuropsychol. Rev. 17:39-59, 2007). Many ADHD susceptibility loci identified by genome linkage or association studies do not contain dopamine-related genes such as dopamine carriers (Franke B. et al., Hum. Genet. 126:13-50, 2009). Therefore, various genetic predispositions are determined to affect this disorder.

Recent genome-wide linkage and association study results in relation to ADHD have reported that several ADHD-susceptibility associated genetic variations may be located in a chromosomal locus comprising of 17q11, 17p11, and 17p12 on human chromosome (Ogdie M. N. et al., Am. J. Hum. Genet. 75:661-668, 2004; Ogdie M N et al., Am. J. Hum. Genet. 72:1268-1279, 2003; Acros-Burgos M et al., Am. J. Hum. Genet. 75:998-1014, 2004). GIT1, which is one of proteins encoded by genes present in this locus, is one of adaptor proteins with a GTPase-activating protein domain, and it is known to regulate various G protein-coupled receptors including β2-adrenergic receptors (Premont R. T. et al., Proc. Natl. Acad. Sci. USA 95: 14082-14087, 1998; Claig A. et al., Proc. Natl. Acad. Sci. USA 97: 1119-1124, 2000). It has been known that, in rodent brains, GIT1 affects various nerve functions, including synapse formation, and in particular, Git1-null mice (Git1^{-/-}) showed impaired dendritic outgrowth, reduced spine density, impaired fear responses and reduced adaptation to new and changing environments (Menon P. et al., Brain Res 1317:218-226, 2010; Schmalzigaug et al., Neurosci. Lett. 458:79-93, 2009).

Therefore, the present inventors noticed these functions of GIT1, and analyzed the association of the GIT1 gene with ADHD susceptibility through a patient-control study, and as a result, first found that a single nucleotide polymorphism (SNP) located in an intron of the GIT1 gene is significantly associated with ADHD susceptibility.

The SNP is a polymorphism with high frequency, which is present on the human genome at a ratio of 0.1% (about 1 nucleotide per 1000 nucleotides). SNP refers to a position in a genome where one nucleotide (or base pair) is substituted with another nucleotide (or base pair), for example, one genome has a C nucleotide while another genome has T nucleotide. In addition, an individual may have only one allele in both copies of autosomal chromosomes (homozygous genotype, for example, T/T), while another individual may have two different alleles in the two autosome copies (heterozygous genotype, for example, C/T). This variation of one nucleotide may cause synthesis of variant amino acids due to variation of codon (missense mutation) or synthesis of a truncated protein due to generation of a stop codon (nonsense mutation). Therefore, it is obvious that presence or absence of the polymorphism is associated with several disorders (Patent Document 1; Korean Patent Registration No. 10-1012601, Patent Document 2; Korean Patent Registration No. 10-1057262), and it has been strongly recognized that it is important to accurately determine presence and absence of polymorphism (SNP typing), for the purpose of diagnosis, genetic screening or treatment, or the like. In addition, SNP is a polymorphism maker that is used when genes related to genetic predisposition to disorders or medicine reactivity are searched for, and receives attention as important genetic information for tailor-made medical treatment.

Therefore, the present inventors identified that the SNP located in an intron of the GIT1 gene can be used to predict genetic disposition for ADHD, and completed the present invention.

### CITED DOCUMENTS

### Patent Documents

(Patent Document 1) Korean Patent No. 10-1012601: Composition for Predicting the Risk of Developing Breast Cancer, Registration Date: 2011/01/27, Applicant: MACROGEN INC
(Patent Document 2) Korean Patent No. 10-1057262: Biomarker for diagnosis of aspirin hypersensitivity, method for manufacturing the same, and method for diagnosis of aspirin hypersensitivity using the same, Registration Date: 2011/08/09, Applicant: Soonchunhyang University Industry Academy Cooperation Foundation

### Non-patent Documents

(Non-patent Document 1) Swanson J. M. et al., Neuropsychol. Rev, 17:39-59, 2007
(Non-patent Document 2) Franke B. et al., Hum. Genet, 126:13-50, 2009
(Non-patent Document 3) Ogdie M. N. et al., Am. J. Hum. Genet. 75:661-668, 2004
(Non-patent Document 4) Ogdie M N et al., Am. J. Hum. Genet. 72:1268-1279, 2003
(Non-patent Document 5) Acros-Burgos M. et al., Am. J. Hum. Genet. 75:998-1014, 2004
(Non-patent Document 6) Premont R. T. et al., Proc. Natl. Acad. Sci. USA 95: 14082-14087, 1998
(Non-patent Document 7) Claig A. et al., Proc. Natl. Acad. Sci. USA 97: 1119-1124, 2000
(Non-patent Document 8) Menon P. et al., Brain Res. 1317:218-226, 2010
(Non-patent Document 9) Schmalzigaug et al., Neurosci. Lett. 458:79-93, 2009

### SUMMARY

An embodiment of the present invention is directed to providing a composition for diagnosing attention deficit hyperactivity disorder (ADHD) risk, including a polynucleotide having C or T at the 24926101st nucleotide residue on the human chromosome 17, which is rs550818 SNP in the GIT1 gene, or a region in the vicinity of the SNP.

Another embodiment of the present invention is directed to providing a composition for diagnosing ADHD risk, including a probe having a chromosomal region that includes a particular SNP having a significant association with ADHD and a complementary sequence, and/or a primer for amplifying the chromosomal region.

Still another embodiment of the present invention is directed to providing a method of predicting the risk of ADHD, by identifying a nucleotide of a particular SNP having a significant association with ADHD with respect to a genetic species taken from a human subject.

Still another embodiment of the present invention is directed to providing a kit for diagnosing the risk of ADHD, including a microarray.

An embodiment of the present invention is directed to providing a method of predicting the risk of attention deficit hyperactivity disorder (ADHD), by identifying or typing the nucleotide of a single nucleotide polymorphism (SNP) in the GIT1 gene having significant association with ADHD, and then afterward measuring the risk of ADHD based on information of the SNP genotypes.

In the present invention, one SNP, rs550818, located at the 24926101st nucleotide residue on the human chromosome 17 (Genome Build 36.3) was identified to have significant association with ADHD risk. In the wild type, nucleotide C is located at the above SNP position, however, when it is changed into nucleotide T due to variation, the variant T dominantly acts in development of ADHD. Hence, when at least one of two alleles is nucleotide T, the risk of ADHD would be significantly increased.

As such, based on the information of the SNP in the present invention, the nucleotide of the SNP is identified from the whole genome of a subject with undeveloped ADHD in order to determine whether or not the risk of ADHD is high in the subject, and thus, the risk of ADHD in the subject can be predicted.

For this reason, one SNP, rs550818, located at the 24926101st nucleotide residue on the human chromosome 17 in the present invention is provided as a composition for diagnosing the risk of ADHD.

Meanwhile, a linkage disequilibrium (hereinafter, LD) region is a region where a particular section on the genome is so short that a cross-over hardly occurs for generations. Therefore, genetic information located in the section is the same and almost conserved for generations. The SNP having a significant association with the risk of ADHD, of the present invention, is a variation present at a particular locus on genome. In addition, polymorphisms are explored where correlation coefficiency (r-square) with the ADHD-associated rs550818 SNP is exhibited 0.95 or more, and polymorphisms are explored where an LD (D′) with the ADHD-associated rs550818 SNP is exhibited 0.95 or more. Therefore, all other variations located in the LD block harboring the rs550818 SNP in the GIT1 gene also have the same genetic information as the SNP, and thus, the LD block harboring the rs550818 SNP in the GIT1 gene can be also provided as a composition for diagnosing ADHD risk.

Based on these, genes of each subject can be analyzed using the composition for diagnosing ADHD risk of the present invention. The composition for diagnosing ADHD risk may include a probe having a chromosomal region that includes a predetermined SNP having a significant association with ADHD and a complementary sequence, and a primer for amplifying the chromosomal region.

A specific method for this gene analysis is not particularly limited, and all the gene detection methods known to the technical fields to which the present invention pertains may be employed. The genetic species may include all the biological species isolated from the subjects. The genetic species is preferably any one selected from the group consisting of, for example, hair, blood, tissues, cells, serum, plasma, saliva, sputum, and urine, and the blood is more preferable, but the genetic species is not limited thereto.

In the above method, the subject means all animals such as a human, a monkey, a dog, a goat, a pig, a mouse, and the like.

The present invention provides a method of predicting the risk of ADHD, the method including:
1) isolating a nucleic acid species from a biological species derived from a subject;
2) identifying the nucleotide of rs550818 single nucleotide polymorphism (SNP) in the GIT1 gene from the nucleic acid isolated from stage 1) at the 24926101st nucleotide residue on human chromosome 17; and
3) determining the risk of ADHD to be high when the alleles or genotype of rs550818 that is identified in stage 2) has one or more T nucleotide.

In the above method, any method known in the art may be used to isolate the nucleic acid from the biological species of the subject. For example, in the case in which nucleic acid of interest is present in a cell, in order to produce pure nucleic acid, first, an extract of the cell is prepared, and then, differential precipitation, column chromatography, extraction with organic solvent, and the like may be further performed. The extract may be prepared using a standard technology in the corresponding art, for example, chemical or mechanical dissolution of a cell. Then, in order to remove any contamination and interference protein, for example, the extract may be subjected to filtration and/or centrifugal separation, and/or may also be treated with chaotropic salts such as guanidium isothiocynate or urea, or organic solvents such as phenyl and/or chloroform. When the chaotropic salt is used, the salt may be preferably removed from the nucleic acid containing species. This procedure may be performed using standard techniques in the art, such as, precipitation, filtration, size exclusion chromatography, and the like. The nucleic acid isolated from the cells or tissues by the above method may be directly purified, or a predetermined region may be specifically amplified using an amplification method such as PCR or real time PCR (RT-PCR) and separated. The nucleic acid includes cDNA synthesized from mRNA as well as DNA. Meanwhile, PCR or RT-PCR may be performed on the whole nucleic acid sequences of the subject, but it may be performed on only a region in the vicinity of an SNP.

In the above method, nucleotide sequencing of the isolated nucleic acid may be performed by various methods known in the art. For example, direct nucleotide sequencing of nucleic acid may be performed by a dideoxy method, or nucleotide sequencing of the SNP region may be performed by hybridizing a probe including a sequence of the SNP region or a probe complementary thereto with the DNA and measuring the hybridization degree obtained therefrom. The hybridization degree may be confirmed by labeling a target DNA with a detectable label to specifically detect only the hybridized target DNA, or by using an electric signal detection method or the like. Specifically, at least one method selected from the group consisting of hybridization by a microarray, allele-specific probe hybridization, allele-specific amplification, sequencing, 5′ nuclease digestion, molecular beacon assay, oligonucleotide ligation assay, size analysis, and single-stranded conformation polymorphism may be performed, but the present invention is not limited thereto.

In the above method, the sequencing of a polynucleotide including a sequence of the SNP region may include hybridizing the nucleic acid species with the polynucleotide immobilized in the microarray; and detecting the hybridization result.

In addition, peptide nucleic acids (PNAs), which are one of the various DNA analogues, may be included as the probe including a sequence of the SNP region. In PNA, a phosphodiester bond of DNA is substituted with a peptide bond and PNA has adenine, thymine, guanine, and cytosine, like DNA, and thus, PNA may bring about a nucleotide-specific hybridizing reaction with DNA and RNA. In particular, unlike natural nucleic acids that electrically repulse each other due to a frame of phosphate bond exhibiting a negative charge, PNA binds to the natural nucleic acid in the hybridizing reaction more strongly than DNA since a frame thereof consists of the peptide bond that does not exhibit an electric charge, and this bond is not affected by salt concentration. In addition, PNA is not degraded by a biodegradable enzyme such as nuclease or protease, and thus, may have higher stability than DNA or RNA. As such, when PNA that enables complementary recognition of the natural nucleic acids and has excellent hybridization bonding strength and stability is labeled with a fluorescent substance, and reacts with the target nucleic acid to occur a hybridizing reaction, a mis-matched portion thereof is degraded and removed by the nuclease, and thereby to be used, as a fluorescence resonance energy transfer (FRET), to analyze the SNP.

Another stage for the sequencing of a polynucleotide including a sequence of the SNP region may include an analysis method capable of improving discrimination power and specificity in detection of nucleotide substitution, insertion, and deletion, by designing the probe where the nucleotide of the SNP complementary to the target gene is positioned at the central portion of the probe and five to six nucleotides are positioned at the left and right thereof, but as a preferable method, a matrix-assisted laser desorption/ionization-time of flight (MALDI-TOF) may be used. The MALDI-TOF may include a method where a matrix molecule is mixed with a biopolymer to be analyzed and then a pulse laser is applied thereto, thereby to ionize a biopolymer.

When laser is applied to the matrix molecule, for example, 3-hydroxypicolinic acid and an analysis material, the matrix molecule absorbs the laser and transmits energy and proton to the analysis material, so that the matrix molecule is ionized. The analysis material is operated by a principle where the mass is analyzed by calculating the amount of time it takes for the analysis material to fly together with the ionized matrix and reach a detector on the opposite side under the vacuum condition. The analysis material having a small amount of mass quickly reaches the detector. Based on the thus obtained mass difference and the sequence of the SNP region that is previously known, the SNP in DNA may be typed.

Meanwhile, the present invention provides a microarray for predicting the risk of ADHD.

The microarray means that the polynucleotide is immobilized in divided regions on a surface of a substrate at a high density, and the regions may be arranged on the substrate at densities of, for example, 400/cm² or higher, 10³/cm², or 10⁴/cm².

The microarray in the present invention preferably includes a polynucleotide hybridizing with C or T nucleotide of the rs550818 SNP in the GIT1 gene, or a complementary polynucleotide thereof, but is not limited thereto. As for the microarray in the present invention, a micropipetting method using a piezoelectric type, a method using a pin type spotter or the like is preferably employed in order to immobilize the probe, used as a probing DNA molecule, on the substrate of the microarray, but the present invention is not limited thereto. The substrate of the microarray is preferably coated with at least one activator selected from the group consisting of amino-silane, poly-L-lysine, and aldehyde, but is not limited thereto. In addition, the substrate may be at least one selected from the group consisting of silicon wafer, glass, quartz, metals, nylon films, nitrocellulose membranes, and plastics, but is not limited thereto. Therefore, the microarray in the present invention may provide a kit for predicting the risk of ADHD.

The kit may include the polynucleotide as a primer and, at the same time, a reagent necessary for amplification.

The kit may further include any one selected from the reactive reagent group consisting of a buffer, reverse transcriptase for synthesizing cDNA from RNA, dNTPs and rNTP (premixing type or separate feeding type), labeling reagents, and washing buffer, which are used in hybridization. Preferably, there may be provided those including a primer for amplifying the region in the vicinity of the rs5508118 SNP in the GIT1 gene.

The primer in the present invention may amplify the SNP region, which is a target portion, and the size thereof and the locus thereof binding to a template are not limited. Any one skilled in the art can easily design the primer by using conventional primer selection software.

The primer has, for example, a nucleotide sequence corresponding to the SNP region, and the 3′ terminal nucleotides of the primer may consist of a complementary sequence (in a specific primer) or a non-complementary sequence (in a non-specific primer) to the nucleotides of the SNP region. The non-specific primer may include non-complementary sequence in other regions as well as the 3′ terminal nucleotides thereof.

The kit in the present invention may be a kit, of which the polynucleotide or a probe derived therefrom is hybridized with nucleic acids in a species and the risk of ADHD for a subject is diagnosed from the hybridized result. In this case, the kit may include the probe and reagents necessary for hybridization. The reagent necessary for hybridization may include for example a hybridizing buffer. The nucleic acids may be amplified or may not be amplified. Therefore, the kit may further include a reagent necessary for amplifying the nucleic acid. The nucleic acid may be labeled with a detectable label. Examples of the detectable label as such may further include any one selected from the group consisting of streptavidin-like phosphatase conjugate, chemifluorescent, and chemiluminescent, and are not limited thereto.

Therefore, the kit in the present invention may include a mismatch probe complementary to the entire region except the SNP region and a perfect match probe complementary to the entire region including the SNP region. The probe may be in a type of microarray such that it is immobilized in a plurality of divided regions on a substrate. When the target sequence is detected in a hybridizing reaction using the perfect match probe and the target sequence is not detected in a hybridizing reaction using the mismatch probe, a sequence associated with ADHD is determined to be present in the species, and the risk of ADHD of the subject is determined from the results.

In the kit, the fluorescent may be used at least one selected from the group consisting of Cy3, Cy5, poly L-lysine-fluorescein isothiocyanate (FITC), rhodamine-B-isothiocyanate (RITC), and rhodamine, but is not limited thereto.

From the above result, the method for predicting the risk of ADHD in the present invention is characterized by analyzing a nucleotide sequence of the genetic species obtained from the human subject, and then, if the nucleotide at the 24926101st nucleotide residue on chromosome 17 corresponds to T, classifying the human subject as a high risk group of ADHD.

### DETAILED DESCRIPTION OF EMBODIMENTS

Hereinafter, the present invention will be described in more detail by specific embodiments. However, the present invention is not limited to the following embodiments, and it is apparent to those skilled in the art that various modifications and changes can be made within the idea and technical scope of the present invention.

Meanwhile, unless technical and scientific terms used herein are defined otherwise, they have meanings understood by those skilled in the art to which the present invention pertains.

In addition, the repetitive descriptions of the same technical constitutions and functions as the prior art will be omitted.

### Example 1

### Study Subjects

The present invention included genomic DNAs of a total of 388 Koreans in order to study an association between an ADHD patient group and a control group. Blood samples of the objects were collected under the approval by the Seoul National University Hospital Institutional Review Board (IRB). Association analysis was performed based on the genotypes of 192 patients with ADHD and 196 age-matched controls without ADHD. Specifically, genome DNAs were extracted from the blood samples obtained from the study subjects using Puregene™ DNA purification kit (Gentra, Minneapolis, Minn.). After the concentration of the extracted DNA was measured using PicoGreen (Molecular Probes, Eugene, Oreg.) fluorescent dye that specifically quantifies only double-stranded DNAs, the blood was stored at a concentration of 2.5 ng/μl suitable for genotyping.

### Example 2

### <2-1> Selection of Gene Polymorphism

The present inventors selected 27 SPNs within a 19-kilobase (kb) region in human chromosome 17 encompassing the GIT1 gene as analysis objects based on the International HapMap Project database, and then analyzed their association with ADHD risk comparing the ADHD patient and control groups.

### <2-2> Genotyping

The GIT1 SNPs were genotyped using the MassARRAY system (Sequenom, San Diego, Calif.) including the matrix assisted laser desorption and ionization-time of flight (MALDI-TOF) mass spectrometry. Sequences of two primers for polymerase chain reaction (PCR) and a primer for nucleotide extension reaction used in the genotyping are shown in Table 1 below, and these primers were designed using the SpectroDESIGN 3.1 program (Sequenom, San Diego, Calif.).

TABLE 1
Sequences of the three primers used for each SNP typing

| | Amplification_ | Amplification_ | |
|---|---|---|---|
| SNP | forward | backward | Extension reaction |
| rs3110496 | ACGTTGGATGCACA | ACGTTGGATGCTCCTGCTT | TCACTGCCGCTCTGGCACC |
| | AATCACTGCCGCTC | GTTAAGATGGG | G |
| | TG | (SEQ ID NO: 28) | (SEQ ID NO: 55) |
| | (SEQ ID NO: 1) | | |
| rs1017529 | ACGTTGGATGCCAG | ACGTTGGATGTATAGTCAG | GATCTGAGGTCTGTGGTCC |
| | GGCAGGATCATAAA | CCTTCCATGCC | TAC |
| | TG | (SEQ ID NO: 29) | (SEQ ID NO: 56) |
| | (SEQ ID NO: 2) | | |
| rs3744626 | ACGTTGGATGTTTC | ACGTTGGATGTCTCAAGGG | GGACCTTTCCGCAGCCTGA |
| | TCCCCCGTGACCTT | TGGAGGAGATG | GTCGTA |
| | TC | (SEQ ID NO: 30) | (SEQ ID NO: 57) |
| | (SEQ ID NO: 3) | | |
| rs3115095 | ACGTTGGATGTTAA | ACGTTGGATGTGTGATGCT | CACTGTTTCTTCATCACAG |
| | CCTCTCTGAGCCAC | GCTTGCAACTG | AGG |
| | TG | (SEQ ID NO: 31) | (SEQ ID NO: 58) |
| | (SEQ ID NO: 4) | | |
| rs1128913 | ACGTTGGATGAAGC | ACGTTGGATGTGTGCACAA | AGACAAAGTCCACCCCATC |
| | AACCCCCAAGACAA | ATGCCAGCATC | AAGT |
| | AG | (SEQ ID NO: 32) | (SEQ ID NO: 59) |
| | (SEQ ID NO:5) | | |
| rs894606 | ACGTTGGATGGCCA | ACGTTGGATGAGTATCTAA | CAATTAATAAACGCTGCCG |
| | ATTAATAAACGCTG | GCAGGGCACAC | TCAGTCA |
| | CC | (SEQ ID NO: 33) | (SEQ ID NO: 60) |
| | (SEQ ID NO: 6) | | |
| rs36053049 | ACGTTGGATGAACA | ACGTTGGATGGCTTCAGGG | AGCGCACGTGCCTAATT |
| | CCTCATTCAACCAG | ATGTGACTAAC | (SEQ ID NO: 61) |
| | CC | (SEQ ID NO: 34) | |
| | (SEQ ID NO: 7) | | |
| rs3785960 | ACGTTGGATGATAC | ACGTTGGATGTAAACCCAG | TAGAATCAGACCATAGAAG |
| | ACATGCACGCATGC | ACCTCTTCCAG | T |
| | AC | (SEQ ID NO: 35) | (SEQ ID NO: 62) |
| | (SEQ ID NO: 8) | | |
| rs4795515 | ACGTTGGATGAGCG | ACGTTGGATGCTGGAAATG | ATTATCTAATCCTCCCTCC |
| | AGACCCCAACTCTT | GAGATGACAGC | C |
| | AA | (SEQ ID NO: 36) | (SEQ ID NO: 63) |
| | (SEQ ID NO: 9) | | |
| rs35481649 | ACGTTGGATGACTC | ACGTTGGATGCCTGCGTCA | GGGCCAGCTTTTGTCGCCC |
| | TCGGGCATTAAAGC | GTCATAAGGG | (SEQ ID NO: 64) |
| | GG | (SEQ ID NO: 37) | |
| | (SEQ ID NO: 10) | | |
| rs41274278 | ACGTTGGATGCTCC | ACGTTGGATGTCTGCAGAG | GCAGGGAGGCCAACACC |
| | TAGCCTCAATAACT | AGACCTAAG | (SEQ ID NO: 65) |
| | GC | (SEQ ID NO: 38) | |
| | (SEQ ID NO: 11) | | |
| rs55956743 | ACGTTGGATGCTCC | ACGTTGGATGTCTGCAGAG | TCCTAGCCTCAATAACTGC |
| | TAGCCTCAATAACT | AGAGACCTAAG | CCCCTG |
| | GC | (SEQ ID NO: 39) | (SEQ ID NO: 66) |
| | (SEQ ID NO: 12) | | |
| rs34131428 | ACGTTGGATGTGCA | ACGTTGGATGTTCTCCGCC | GACAGCACTTTGCACCC |
| | GCGAGAGGTGAGGG | TGCAGCTTGTG | (SEQ ID NO: 67) |
| | T | (SEQ ID NO: 40) | |
| | (SEQ ID NO: 13) | | |
| rs11548561 | ACGTTGGATGAAAG | ACGTTGGATGAGTGAACGG | AGGGCAGAGCCAGGTTCA |
| | GGCTTCAGGGCAGA | GCGGAACACA | (SEQ ID NO: 68) |
| | G | (SEQ ID NO: 41) | |
| | (SEQ ID NO: 14) | | |
| ENSSNP11225832 | ACGTTGGATGAAAG | ACGTTGGATGAGTGAACGG | GGCGGAACACACACCCAT |
| | GGCTTCAGGGCAGA | GCGGAACACA | (SEQ ID NO: 69) |
| | G | (SEQ ID NO: 42) | |
| | (SEQ ID NO: 15) | | |
| rs34015437 | ACGTTGGATGAGCG | ACGTTGGATGCACACTGAC | AGCTGAGGCAGACACCCCT |
| | AAGGGCACAGCTGA | TCCATCCAGC | TT |
| | G | (SEQ ID NO: 43) | (SEQ ID NO: 70) |
| | (SEQ ID NO: 16) | | |
| rs36109144 | ACGTTGGATGGGTG | ACGTTGGATGGTCTCTGTT | AACCTCTTCCCTTCCAG |
| | GAAGTCTTCCTCTT | TTCTGGACCTG | (SEQ ID NO: 71) |
| | TG | (SEQ ID NO: 44) | |
| | (SEQ ID NO: 17) | | |
| rs550818 | ACGTTGGATGTGAG | ACGTTGGATGAATCCCATC | CCAACCTTGGACTTTAAGC |
| | GCTGGACCTTGGAC | TGATCGCTACC | C |
| | TT | (SEQ ID NO: 45) | (SEQ ID NO: 72) |
| | (SEQ ID NO: 18) | | |
| rs55778351 | ACGTTGGATGGAGG | ACGTTGGATGTTAGTGCTG | ACGTTGGATGTTAGTGCTG |
| | TGCATGGAATGTGG | CCACACTCCCT | CCACACTCCCT |
| | G | (SEQ ID NO: 46) | (SEQ ID NO: 73) |
| | (SEQ ID NO: 19) | | |
| rs41274276 | ACGTTGGATGACCC | ACGTTGGATGGGAGACTAT | CCCATGAGCCAGTTCAGCC |
| | CCATGAGCCAGTTC | GTACAAAGGAG | CTAC |
| | AG | (SEQ ID NO: 47) | (SEQ ID NO: 74) |
| | (SEQ ID NO: 20) | | |
| rs3211044 | ACGTTGGATGTGCG | ACGTTGGATGATCAGGGAG | GGACTGTCCGGGTAGCCAG |
| | TGTTGAGTGTGTGG | GCAGCTGGC | TTT |
| | G | (SEQ ID NO: 48) | (SEQ ID NO: 75) |
| | (SEQ ID NO: 21) | | |
| rs57467547 | ACGTTGGATGTGCG | ACGTTGGATGATCAGGGAG | AGCTTCCCTTCCCCACC |
| | TGTTGAGTGTGTGG | GCAGCTGGC | (SEQ ID NO: 76) |
| | G | (SEQ ID NO: 49) | |
| | (SEQ ID NO: 22) | | |
| rs11548559 | ACGTTGGATGTGCG | ACGTTGGATGATCAGGGAG | GATGGACCAGGCCTCCGGA |
| | TGTTGAGTGTGTGG | GCAGCTGGC | A |
| | G | (SEQ ID NO: 50) | (SEQ ID NO: 77) |
| | (SEQ ID NO: 23) | | |
| rs60006555 | ACGTTGGATGTGCG | ACGTTGGATGACCCTAGCC | GAATCAAAAACCACTTCCC |
| | CCCCTTCTCTATGA | AGCACACATTC | TCC |
| | AC | (SEQ ID NO: 51) | (SEQ ID NO: 78) |
| | (SEQ ID NO: 24) | | |
| rs55677368 | ACGTTGGATGACCC | ACGTTGGATGATCACTACC | CCCAGGGAAGAAGCGGGAT |
| | ACCACCACACAGGT | TGCACCTGCTG | (SEQ ID NO: 79) |
| | AG | (SEQ ID NO: 52) | |
| | (SEQ ID NO: 25) | | |
| rs11548562 | ACGTTGGATGACCC | ACGTTGGATGATCACTACC | ACAGCCTTCCCAGGCTGC |
| | ACCACCACACAGGT | TGCACCTGCTG | (SEQ ID NO: 80) |
| | AG | (SEQ ID NO: 53) | |
| | (SEQ ID NO: 26) | | |
| rs565977 | ACGTTGGATGCGAG | ACGTTGGATGGGGAATGGA | CGAGGAAGGTGGGGACAA |
| | AGGAGGTAACTGTT | AAATTCAAGGG | (SEQ ID NO: 81) |
| | AG | (SEQ ID NO: 54) | |
| | (SEQ ID NO: 27) | | |

The PCR was performed using 5 μl of an aqueous solution where 200 nM of each PCR primer of Table 1 was added to 2.5 ng of genome DNA, 1× buffer, 1 mM of MgCl₂, 200 μM of dNTP mixture, and 0.1 unit of HotStart Taq polymerase mixture. The PCR was run under the reaction conditions of an initial denaturation stage of 94° C. for 15 minutes, followed by 45 cycles of 94° C. for 20 seconds, 56° C. for 30 seconds, and 72° C. for 1 minute, and a final elongation stage of 72° C. for 3 minutes. After the PCR, 0.3 units of shrimp alkaline phosphatase (SAP) was added, followed by incubation at 37° C. for 20 minutes and 85° C. for 5 minutes, thereby removing residual dNTPs. The nucleotide extension reaction was performed by adding 50 μM of dNTP terminator mix, 625 nM of extension primer mix, and 0.5 units of Thermosequenase enzyme to the resultant reaction. After initial denaturation was performed at 94° C. for 30 seconds, 40 cycles of 94° C. for 5 seconds, 52° C. for 5 seconds, and 80° C. for 5 seconds were repeated. After 16 μl of distilled water and 3 mg of clean resin were added thereto, the final reaction product was transferred onto SpectroChip (Sequenom, San Diego, Calif.), and then genotyping was done using mass spectrometry. As a genotyping result of 27 SNPs in Example 1, Table 2 shows that only 8 SNPs exhibited polymorphism (heterozygosity>0) in the patient and control groups.

TABLE 2
Genotype frequency of SNP in GIT1

| | Position | | | | |
|---|---|---|---|---|---|
| | on chro- | | Major | Minor | Hetero- |
| SNP ID | mosome | Location | allele | allele | zygosity |
| rs3110496 | 24941897 | Promoter | 300 | 92 | 0.36 |
| rs1017529 | 24936541 | Intron 1 | 362 | 30 | 0.14 |
| rs3744626 | 24935683 | Intron 1 | 247 | 145 | 0.47 |
| rs3115095 | 24935404 | Intron 1 | 359 | 33 | 0.15 |
| rs1128913 | 24933936 | Exon 4 | 384 | 0 | 0 |
| rs894606 | 24933478 | Intron 4 | 255 | 137 | 0.45 |
| rs36053049 | 24931747 | Intron 7 | 392 | 0 | 0 |
| rs3785960 | 24931466 | Intron 7 | 346 | 46 | 0.21 |
| rs4795515 | 24930925 | Intron 7 | 392 | 0 | 0 |
| rs35481649 | 24928389 | Exon 12 | 392 | 0 | 0 |
| rs41274278 | 24927873 | Intron 13 | 390 | 0 | 0 |
| rs55956743 | 24927872 | Intron 13 | 392 | 0 | 0 |
| rs34131428 | 24927598 | Intron 14 | 392 | 0 | 0 |
| rs11548561 | 24927496 | Exon 15 | 392 | 0 | 0 |
| ENSSNP11225832 | 24927446 | Exon 15 | 392 | 0 | 0 |
| rs34015437 | 24927033 | Exon 17 | 392 | 0 | 0 |
| rs36109144 | 24926614 | Exon 19 | 392 | 0 | 0 |
| rs550818 | 24926101 | Intron 20 | 374 | 18 | 0.09 |
| rs55778351 | 24925677 | 3′ UTR | 392 | 0 | 0 |
| rs41274276 | 24925454 | 3′ UTR | 392 | 0 | 0 |
| rs3211044 | 24925184 | 3′ UTR | 392 | 0 | 0 |
| rs57467547 | 24925095 | 3′ UTR | 392 | 0 | 0 |
| rs11548559 | 24925064 | 3′ UTR | 392 | 0 | 0 |
| rs60006555 | 24924984 | 3′ UTR | 392 | 0 | 0 |
| rs55677368 | 24924775 | 3′ UTR | 392 | 0 | 0 |
| rs11548562 | 24924722 | 3′ UTR | 392 | 0 | 0 |
| rs565977 | 24922954 | Down- | 295 | 97 | 0.37 |
| | | stream | | | |

### Example 3

### Statistical Analysis

In the statistical analysis of the present invention, Hardy-Weinberg equilibrium (HWE) was analyzed using a chi-square test for independence, and association between the risk of ADHD and each polymorphism was tested using logistic regression analysis with adjustment for gender and IQ score in comparisons between the ADHD patient group and the control group. All statistical analyses were done using the SPSS 15 program.

### Example 4

### Association with ADHD Susceptibility

Association analysis with ADHD susceptibility for 27 SNPs in the GIT1 gene of the present invention was done.

As the result, as shown in Table 3, the risk of ADHD was higher by about 2.66 times in the subjects carrying the heterozygote C/T genotype at the rs550818 SNP than those carrying the homozygote C/C genotype (OR=2.66, 95% CI=1.33-5.31, P=0.0056). Therefore, as a result of association analysis with ADHD susceptibility using the SNP genotypes, it was confirmed that the heterozygote C/T genotype or carriage of T nucleotide allele in the rs550818 SNP has strong association with an increase in the risk of ADHD in comparison with homozygote CC genotype or non-carriage of T nucleotide.

TABLE 3
Association of rs550818 SNP in GIT1 with the risk of ADHD

| | Patient | Control | | |
|---|---|---|---|---|
| | group | group | OR | |
| Geno-type | (n = 192) | (n = 196) | (95% CI) | P |
| CC | 155 (80.7%) | 178 (90.8%) | 1 | |
| CT | 36 (18.8%) | 18 (9.2%) | 2.66 (1.33-5.31) | 0.0056 |
| TT | 1 (0.5%) | 0 (0%) | — | — |

The rs550818 SNP marker in the GIT1 gene has a significant association with ADHD susceptibility, and thus, the SNP can be used to constitute the composition and the kit for diagnosing the risk of ADHD by SNP analysis.

Further, according to the method of predicting the risk of ADHD of the present invention, the analysis of genetic information (SNP) and research on clinical factors such as gender, IQ scores, and the like, are performed in parallel, so that accuracy can be further improved.

What is claimed is:

1. A method for predicting the risk of attention deficit hyperactivity disorder (ADHD), the method comprising:
1) isolating a nucleic acid species from a biological species derived from a subject;
2) identifying the nucleotide of rs550818 single nucleotide polymorphism (SNP) in the GIT1 gene, which is the 24926101st nucleotide residue on human chromosome 17, from the nucleic acid isolated from stage 1); and
3) determining the risk of ADHD to be high when the genotype of rs550818 SNP that is identified in stage 2) carries T nucleotide;
Wherein the composition comprising a primer pair for amplifying a rs550818 single nucleotide polymorphism (SNP), wherein the primer pair comprises a primer having a sequence of SEQ ID NO:18 and a primer having a sequence of SEQ ID NO: 45.

2. The method of claim 1, wherein the biological species is at least one selected from the group consisting of hair, blood, tissues, cells, serum, plasma, saliva, sputum, and urine.

3. The method of claim 1, wherein the stage 2) is performed by at least one method selected from the group consisting of hybridization by microarrays, allele-specific probe hybridization, allele-specific amplification, sequencing, 5′ nuclease digestion, molecular beacon assay, oligonucleotide ligation assay, size analysis, and single-stranded conformation polymorphism.

4. A method for predicting the risk of attention deficit hyperactivity disorder (ADHD), the method comprising determining a single nucleotide polymorphism (SNP) by a polymerase chain reaction (PCR) using a probe having a sequence of the rs550818 SNP in the GIT1 gene, a primer, or both of the probe and the primer.

5. The method of claim 4, wherein the PCR is a real time PCR (RT-PCR) or a PCR using a PNA probe.
